(19) United States
(12) Patent Application Publication
(10) Pub. No.: US 2012/0059247 A1
(43) Pub. Date: Mar. 8, 2012

(54) ECHOGENIC NEEDLE FOR BIOPSY DEVICE

(76) Inventors: Trevor W.V. Speeg, Williamsburg, OH (US); Matthew C. Miller, Cincinnati, OH (US); Michael J. Vendely, Lebanon, OH (US); Lucia G. Buehler, Loveland, OH (US); Edward A. Rhad, Fairfield, OH (US); Kirk S. Leonard, Huntington Woods, MI (US); Daniel H. Duke, Franklin, OH (US); Frederick E. Shelton, IV, Hillsboro, OH (US)

(21) Appl. No.: 12/875,200

(22) Filed: Sep. 3, 2010

(51) Int. Cl. A61B 10/02; A61B 8/00

(52) U.S. Cl. 600/424; 600/567

(57) ABSTRACT

A biopsy device comprises an elongate needle having a piercing tip, a lateral aperture, and one or more echogenic features. In some versions, a dimpled surface provides the one or more echogenic features. The dimples may be concave or convex. In some versions, the piercing tip comprises a blade, and the one or more echogenic features are provided by openings formed transversely through the blade. In some versions, the one or more echogenic features are provided by serrations of the blade. Such serrations may be jagged or rounded. In some versions, the piercing tip is multi-faceted, and the facets of the tip provide the one or more echogenic features. A coagulant may also be provided on the needle. The piercing tip of the needle may rotate relative to other portions of the needle to facilitate insertion of the needle in tissue.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Merely exemplary biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2003/0109803, entitled “MRI Compatible Surgical Biopsy Device,” published Jun. 12, 2003; U.S. Pub. No. 2006/0074345, entitled “Biopsy Apparatus and Method,” published Apr. 6, 2006; U.S. Pub. No. 2007/0118048, entitled “Remote Thumbwheel for a Surgical Biopsy Device,” published May 24, 2007; U.S. Pub. No. 2008/0214955, entitled “Presentation of Biopsy Sample by Biopsy Device,” published Sep. 4, 2008; U.S. Pub. No. 2009/0171242, entitled “Clutch and Valving System for Tetherless Biopsy Device,” published Jul. 2, 2009; U.S. Pub. No. 2010/0152610, entitled “Hand Actuated Tetherless Biopsy Device with Pistol Grip,” published Jun. 17, 2010; U.S. Pub. No. 2010/0160819, entitled “Biopsy Device with Central Thumbwheel,” published Jun. 24, 2010; U.S. non-provisional patent application Ser. No. 12/483,305, entitled “Tetherless Biopsy Device with Reusable Portion,” filed Jun. 12, 2009; and U.S. non-provisional patent application Ser. No. 12/709,624, entitled “Spring Loaded Biopsy Device,” filed Feb. 22, 2010. The disclosure of each of the above-cited U.S. Patents, U.S. Patent Application Publications, and U.S. Non-Provisional Patent Applications is incorporated by reference herein.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an exemplary biopsy device.

FIG. 2 depicts a block schematic view of components that are part of, or used with, the device of FIG. 1.

FIG. 3 depicts a first series view of part of the needle of the biopsy device of FIG. 1, with the needle shown in cross section and with the cutter in the initial, distal position.

FIG. 4 depicts a second series view of part of the needle of the biopsy device of FIG. 1, with the needle shown in cross section and with the cutter in an intermediate position during retraction.

FIG. 5 depicts a third series view of part of the needle of the biopsy device of FIG. 1, with the needle shown in cross section and with the cutter in the retracted, proximal position.

FIG. 6 depicts a fourth series view of part of the needle of the biopsy device of FIG. 1, with the needle shown in cross section and with the cutter in the advanced, distal position.

FIG. 7 depicts a partial perspective view of an exemplary alternative version of the needle of the biopsy device of FIG. 1, having dimples on the surface of the needle.

FIG. 8 depicts a partial side view of another exemplary alternative version of the needle of the biopsy device of FIG. 1, having dimples on the surface of the needle and a serrated distal edge.

FIG. 9 depicts a partial side view of yet another exemplary alternative version of the needle of the biopsy device of FIG. 1, having either protuberances or openings associated with its distal blade.

FIG. 10 depicts a partial side view of yet another exemplary alternative version of the needle of the biopsy device of FIG. 1, having scalloped sides and a diamond faceted tip.

FIG. 11 depicts a partial side view of yet another exemplary alternative version of the needle of the biopsy device of FIG. 1, having dimples on the tip of the needle and a serrated distal edge.

FIG. 12A depicts a partial side view of yet another exemplary alternative version of the needle of the biopsy device of FIG. 1, having a cutter with a grooved surface.

FIG. 12B depicts a partial perspective view of the cutter of FIG. 12A.

FIG. 13 depicts a partial perspective view of yet another exemplary alternative version of the needle of the biopsy device of FIG. 1, having an outer coating on the needle.

FIG. 14 depicts a partial perspective view of yet another exemplary alternative version of the needle of the biopsy device of FIG. 1, having a rotating needle tip.

FIG. 15 depicts a partial side view of yet another exemplary alternative version of the needle of the biopsy device of FIG. 1, having a blade with oblong serrations.

FIG. 16 depicts a partial top view of the needle of FIG. 15.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Overview

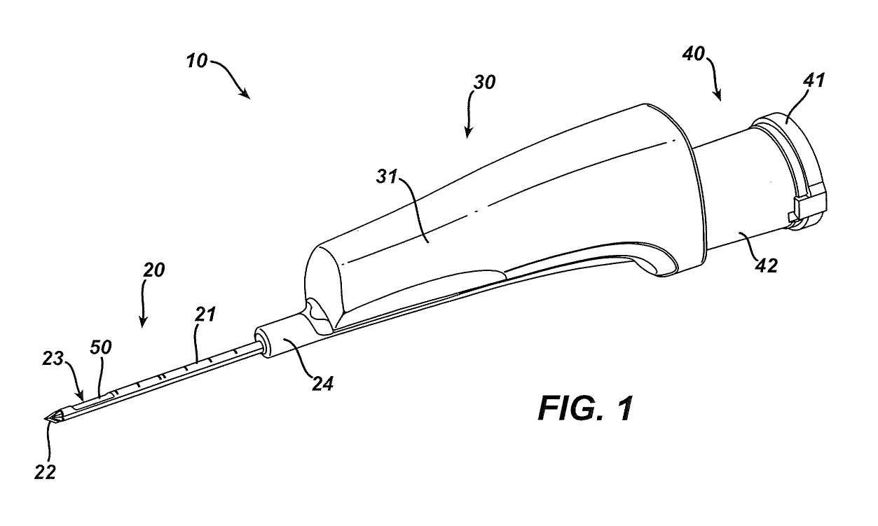

As shown in FIG. 1, an exemplary biopsy device (10) comprises a needle (20), a body (30), a tissue sample holder (40), and a cutter (50). In particular, needle (20) extends distally from the distal portion of body (30), while tissue sample holder (40) extends proximally from the proximal portion of body (30). Body (30) is sized and configured such that biopsy device (10) may be operated by a single hand of a user. In particular, a user may grasp body (30), insert needle (20) into a patient's breast, and collect one or a plurality of tissue samples from within the patient's breast, all with just using a single hand. Alternatively, a user may grasp body (30) with more than one hand and/or with any desired assistance. In some settings, the user may capture a plurality of tissue samples with just a single insertion of needle (20) into the patient's breast. Such tissue samples may be pneumatically deposited in tissue sample holder (40), and later retrieved from tissue sample holder (40) for analysis. While examples described herein often refer to the acquisition of biopsy samples from a patient's breast, it should be understood that biopsy device (10) may be used in a variety of other procedures for a variety of other purposes and in a variety of other parts of a patient's anatomy.

Needle (20) of the present example comprises a cannula (21) with a tissue piercing tip (22), a lateral aperture (23), and a hub (24). Tissue piercing tip (22) is configured to pierce and penetrate tissue, without requiring a high amount of force, and without requiring an opening to be pre-formed in the tissue prior to insertion of tip (22). Alternatively, tip (22) may be blunt (e.g., rounded, flat, etc.) if desired. Lateral aperture (23) is sized to receive a tissue from a tissue specimen during operation of device (10). Within cannula (21) resides cutter (50), which rotates and translates relative to cannula (21) and past lateral aperture (23) to sever a tissue sample from tissue protruding through lateral aperture (23). Hub (24) may be formed of plastic that is overmolded about needle (20) or otherwise secured to needle (20), such that hub (24) is unitarily secured to needle (20). Alternatively, hub (24) may be formed of any other suitable material through any suitable process and may have any other suitable relationship with needle (20). Hub (24) of the present example is coupled with a vacuum conduit (not shown), and is operable to communicate a vacuum (or atmospheric air, saline, pressurized fluid, etc.) from vacuum conduit to lateral aperture (23). The vacuum conduit may be coupled with a variety of sources, including but not limited to a vacuum source that is internal or external to biopsy device (10) in accordance with the teachings of U.S. non-provisional patent application Ser. No. 12/483,305, entitled “Tetherless Biopsy Device with Reusable Portion,” filed Jun. 12, 2009, and/or U.S. Pub. No. 2008/0214955, entitled “Presentation of Biopsy Sample by Biopsy Device,” published Sep. 4, 2008, the disclosures of which are incorporated by reference herein. Still other suitable fluid sources that a vacuum conduit may be coupled with will be apparent to those of ordinary skill in the art in view of the teachings herein. Of course, any suitable type of valve(s) and/or switching mechanism(s) may also be coupled with vacuum conduit, e.g., as taught in U.S. non-provisional patent application Ser. No. 12/483,305, entitled “Tetherless Biopsy Device with Reusable Portion,” filed Jun. 12, 2009, and/or U.S. Pub. No. 2008/0214955, entitled “Presentation of Biopsy Sample by Biopsy Device,” published Sep. 4, 2008, the disclosures of which are incorporated by reference herein. It should also be understood that a vacuum, atmospheric air, a liquid such as saline, etc. may also be selectively communicated to the lumen defined by cutter (50).

Body (30) of the present example comprises a housing (31). In some versions, body (30) is formed in at least two pieces, comprising a probe portion and a holster portion. For instance, in some such versions, the probe portion may be separable from the holster portion. Furthermore, the probe portion may be provided as a disposable component while the holster portion may be provided as a reusable portion. By way of example only, such a probe and holster configuration may be provided in accordance with the teachings of U.S. non-provisional patent application Ser. No. 12/483,305, entitled “Tetherless Biopsy Device with Reusable Portion,” filed Jun. 12, 2009, and/or U.S. Pub. No. 2008/0214955, entitled “Presentation of Biopsy Sample by Biopsy Device,” published Sep. 4, 2008, the disclosures of which are incorporated by reference herein. Alternatively, any other suitable probe and holster configuration may be used. It should also be understood that body (30) may be configured such that it does not have a separable probe portion and holster portion. Various other suitable ways in which body (30) may be configured will be apparent to those of ordinary skill in the art in view of the teachings herein.

Tissue sample holder (40) of the present example comprises a cap (41) and an outer cup (42). A filter tray (not shown) is provided within outer cup (42). Outer cup (42) is secured to body (30) in the present example. Such engagement may be provided in any suitable fashion. Outer cup (42) of the present example is substantially transparent, allowing the user to view tissue samples on the filter tray, though outer cup (42) may have any other suitable properties if desired. The hollow interior of outer cup (42) is in fluid communication with cutter (50) and with a vacuum source in the present example. By way of example only, vacuum may be provided to outer cup (42), and such a vacuum may be further communicated to cutter (50), in accordance with the teachings of U.S. non-provisional application Ser. No. 12/483,305, entitled “Tetherless Biopsy Device with Reusable Portion,” filed Jun. 12, 2009, and/or U.S. Pub. No. 2008/0214955, entitled “Presentation of Biopsy Sample by Biopsy Device,” published Sep. 4, 2008, the disclosures of which are incorporated by reference herein. Various other suitable ways in which vacuum may be provided to outer cup (42) will be apparent to those of ordinary skill in the art in view of the teachings herein. It should also be understood that outer cup (42) may receive vacuum from the same vacuum source as the vacuum conduit in needle (20). Biopsy device (10) may further include one or more valves (e.g., shuttle valve, electromechanical solenoid valve, etc.) to selectively regulate communication of a vacuum and/or other fluids to outer cup (42) and/or vacuum conduit, regardless of whether outer cup (42) and vacuum conduit are coupled with a common source of vacuum or other source of fluid.

In the present example, when a tissue sample has been severed from a tissue specimen by cutter (50), the tissue sample is pulled from cutter (50) to tissue sampler holder (40) by the vacuum. Cap (41) is removably coupled with outer cup (42) in the present example such that a user may remove cap (41) to access tissue samples that have gathered on the filter tray (not shown) within outer cup (42) during a biopsy process. In lieu of having a stationary filter tray, tissue sample holder (40) may have a plurality of trays that are removably coupled with a rotatable manifold, such that the manifold is operable to successively index each tray relative to cutter (50) to separately receive tissue samples obtained in successive cutting strokes of cutter (50). For instance, tissue sample holder (40) may be constructed and operable in accordance with the teachings of U.S. Pub. No. 2008/0214955, entitled “Presentation of Biopsy Sample by Biopsy Device,” published Sep. 4, 2008, the disclosure of which is incorporated by reference herein. As another merely illustrative example, tissue sample holder (40) may be constructed and operable in accordance with the teachings of U.S. non-provisional application Ser. No. 12/337,911, entitled “Biopsy Device with Discrete Tissue Chambers,” filed Dec. 18, 2008. Still other suitable ways in which tissue sample holder (40) may be constructed and operable will be apparent to those of ordinary skill in the art in view of the teachings herein.

It should be understood that, as with other components described herein, needle (20), body (30), tissue sample holder (40), and cutter (50) may be varied, modified, substituted, or supplemented in a variety of ways, and that needle (20), body (30), tissue sample holder (40), and cutter (50) may have a variety of alternative features, components, configurations, and functionalities. Several merely exemplary variations, modifications, substitutions, or supplementations are described in U.S. non-provisional patent application Ser. No. 12/709,624, entitled “Spring Loaded Biopsy Device,” filed Feb. 22, 2010, the disclosure of which is hereby incorporated by reference. Still yet, other suitable alternative versions, features, components, configurations, and functionalities of needle (20), body (30), tissue sample holder (40), and cutter (50) will be apparent to those of ordinary skill in the art in view of the teachings herein.

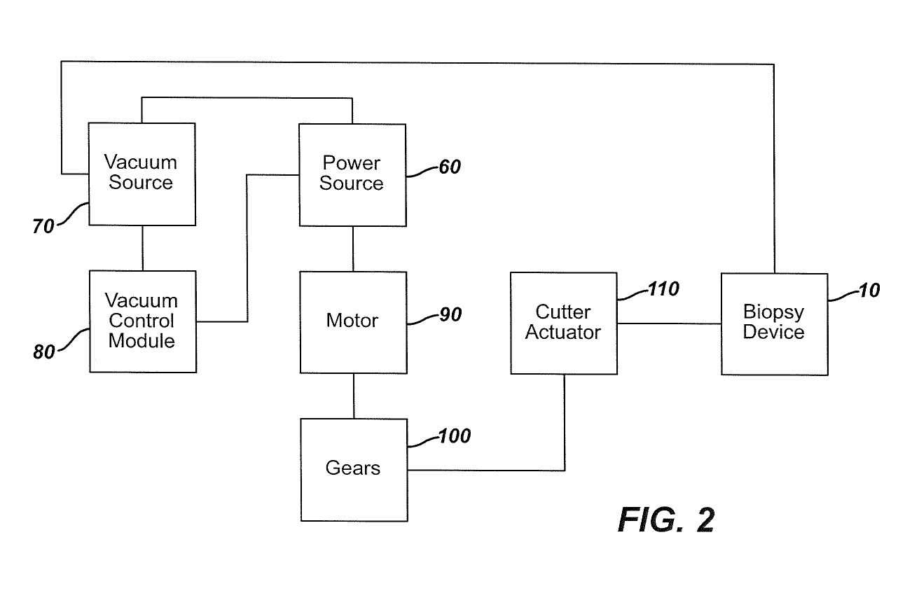

As shown in FIG. 2, exemplary components that are part of, or used with, the device of FIG. 1, some of which have been introduced above, include a power source (60), a vacuum source (70), a vacuum control module (80), a motor (90), a set of gears (100), and a cutter actuator (110). In the present example, power source (60) provides power to vacuum source (70), vacuum control module (80), and motor (90). In some versions, power source (60) is located onboard biopsy device (10), e.g., a battery; while in some other versions, power source (60) is located some distance from biopsy device (10), e.g., line voltage from a standard electrical receptacle with a cable connection to biopsy device (10) and/or through an additional module between an electrical receptacle and biopsy device (10). Various configurations for and modifications to power source (60) will be apparent to those of ordinary skill in the art in view of the teachings herein.

In the present example, vacuum source (70) provides vacuum to biopsy device (10) for drawing tissue into lateral aperture (23) of needle (20). Vacuum source (70) also provides vacuum to biopsy device (10) for transporting a severed tissue sample from cutter (50) to tissue sample holder (40). In some versions, vacuum source (70) comprises a vacuum pump located onboard biopsy device (10). By way of example only, such an onboard vacuum source (70) may comprise a diaphragm pump that is driven by motor (90). In some such versions, vacuum source (70) is not coupled with power source (60) and vacuum control module (80) is omitted. In some other versions, vacuum source (70) comprises a vacuum pump located some distance from biopsy device (10) that provides vacuum via a vacuum cable or conduit. Of course, vacuum source (70) may comprise a combination of a vacuum pump located within housing (31) and a vacuum pump that is external to housing (31), if desired. In the present example, vacuum source (70) is in communication with vacuum control module (80). Vacuum control module (80) includes functions to control the supply and delivery of vacuum from vacuum source (70) to biopsy device (10). Various functions and capabilities that can be used with vacuum control module (80) to control how vacuum is supplied and delivered will be apparent to those of ordinary skill in the art in view of the teachings herein. Also, various other configurations for, and modifications to, vacuum source (70) and vacuum control module (80) will be apparent to those of ordinary skill in the art based on the teachings herein.

Motor (90) of the present example comprises a conventional DC motor, though it should be understood that any other suitable type of motor may be used. By way of example only, motor (90) may comprise a pneumatic motor (e.g., having an impeller, etc.) that is powered by pressurized air, a pneumatic linear actuator, an electromechanical linear actuator, a piezoelectric motor (e.g., for use in MRI settings), or a variety of other types of movement-inducing devices. As mentioned above, motor (90) receives power from power source (60). In some versions, motor (90) is located onboard biopsy device (10) (e.g., within housing (31)). In some other versions, motor (90) is located some distance from biopsy device (10) and provides energy to biopsy device (10) via a drive shaft or cable. In the present example, motor (90) is operable to rotate a drive shaft (not shown), which extends distally from motor (90) to gear set (100) to provide a rotary input into gear set (100). While the drive shaft extends directly from motor (90) into gear set (100), it should be understood that a variety of other components may be coupled between motor (90) and gear set (100), including but not limited to various gears, a clutch, etc. Gear set (100) includes an output shaft (not shown) having a drive gear (not shown) secured thereto, and is operable to selectively activate cutter actuator (110). Gear set (100) may comprise a planetary gearbox, and may be configured to provide speed reduction. Various suitable configurations for motor (90) and gear set (100) will be apparent to those of ordinary skill in the art in view of the teachings herein.

Cutter actuator (110) of the present example comprises a variety of components that interact to provide simultaneous rotation and distal translation of cutter (50) relative to body (30) and needle (20) in a firing stroke. Cutter actuator (110) is also operable to retract cutter (50) proximally to ready cutter (50) for firing. By way of example only, cutter actuator (110) may be configured and operable in accordance with the teachings of U.S. non-provisional patent application Ser. No. 12/709,624, entitled “Spring Loaded Biopsy Device,” filed Feb. 22, 2010, and/or U.S. Pub. No. 2008/0214955, entitled “Presentation of Biopsy Sample by Biopsy Device,” published Sep. 4, 2008, the disclosures of which are incorporated by reference herein. It should be understood that, as with other components described herein, cutter actuator (110) may be varied, modified, substituted, or supplemented in a variety of ways, and that cutter actuator (110) may have a variety of alternative features, components, configurations, and functionalities. Suitable alternative versions, features, components, configurations, and functionalities of cutter actuator (110) will be apparent to those of ordinary skill in the art in view of the teachings herein.

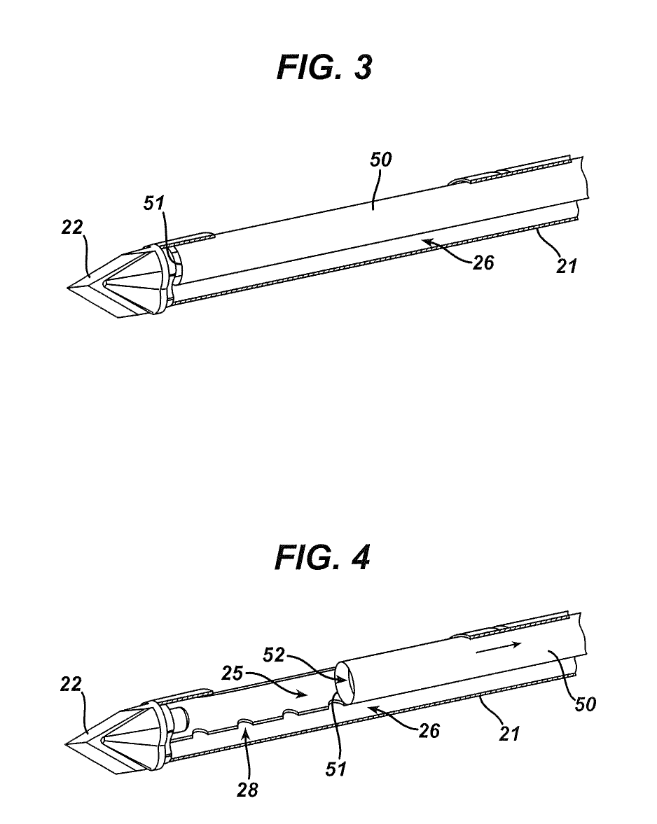

As shown in the series views of FIGS. 3-6, an exemplary cutter (50) firing sequence is shown. FIG. 3 depicts cutter (50) in a distal position, with distal edge (51) of cutter (50) positioned distal of lateral aperture (23) thereby effectively “closing” lateral aperture (23) of needle (20). In this configuration, needle (20) can be inserted without tissue prolapsing through lateral aperture (23). FIG. 4 depicts cutter (50) being retracted by cutter actuator (110), thereby exposing tissue to lateral aperture (23) and revealing a cutter lumen (52) of cutter (50). In the present example, cutter (50) is positioned within a first lumen (25) of cannula (21). Beneath first lumen (25) is a second lumen (26), which is in part defined by a divider (27). Divider (27) comprises a plurality of openings (28) that provide fluid communication between first and second lumens (25, 26). A plurality of external openings (not shown) may also be formed in needle (20), and may be in fluid communication with second lumen (26). For instance, such external openings may be configured in accordance with the teachings of U.S. Pub. No. 2007/0032742, entitled “Biopsy Device with Vacuum Assisted Bleeding Control,” published Feb. 8, 2007, the disclosure of which is incorporated by reference herein. Cutter (50) may also include one or more side openings (not shown). Of course, as with other components described herein, such external openings in needle (20) and cutter (50) are merely optional.

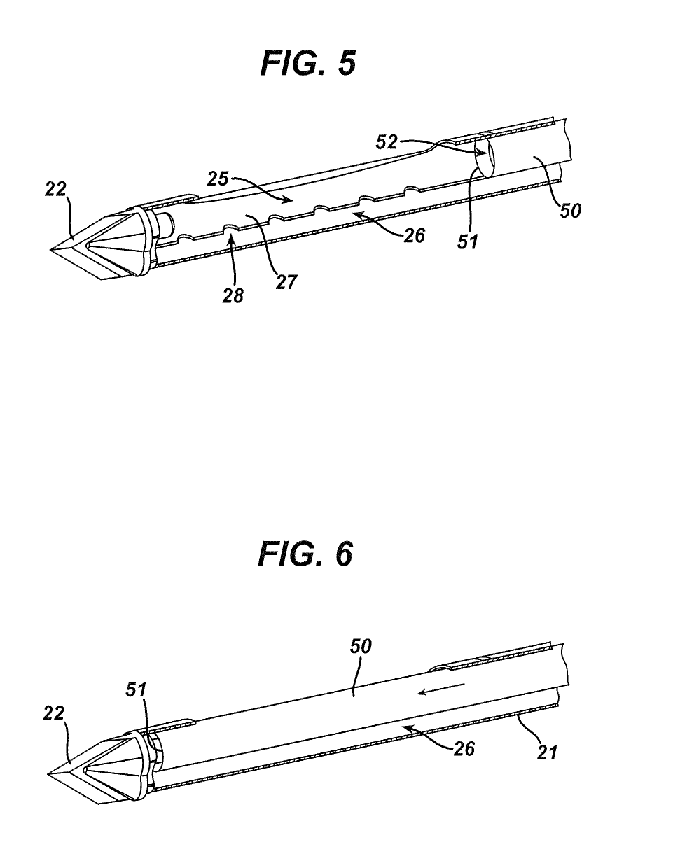

FIG. 5 depicts cutter (50) fully retracted by cutter actuator (110), such that lateral aperture (23) is completely unobstructed by cutter (50). In this configuration tissue can prolapse through lateral aperture (23) within first lumen (25) under the force of gravity, due to internal pressure of the tissue (e.g., caused by displacement of the tissue upon insertion of needle (20), etc.), and/or with vacuum provided through second lumen (26) and transmitted through openings (28) and/or by vacuum provided through cutter lumen (52). FIG. 6 depicts cutter (50) after it has been advanced to close off lateral aperture (23) once tissue has been captured within first lumen (25). With the tissue severed, it is captured within cutter lumen (52) and ready for proximal transport to tissue sample holder (40). Such proximal transport of tissue through cutter lumen (52) to reach tissue sample holder (40) may be provided by drawing a vacuum through the proximal portion of cutter lumen (52) (e.g., behind the captured tissue sample) while venting a distal portion of cutter lumen (52) (e.g., in front of the captured tissue sample) to provide a pressure differential. Alternatively, tissue samples severed by cutter (50) may be communicated proximally to tissue sample holder (40) or be otherwise dealt with in any other suitable fashion.

While the above paragraphs provide an enabling description of an exemplary biopsy device (10) and its use, further description as well as exemplary methods of operation are provided with the teachings of U.S. non-provisional patent application Ser. No. 12/709,624, entitled “Spring Loaded Biopsy Device,” filed Feb. 22, 2010, and U.S. Pub. No. 2008/0214955, entitled “Presentation of Biopsy Sample by Biopsy Device,” published Sep. 4, 2008, the disclosures of which are incorporated by reference herein. Of course, the above examples of construction and use of biopsy device (10) are merely illustrative. Other suitable ways in which biopsy device (10) may be made and used will be apparent to those of ordinary skill in the art in view of the teachings herein.

II. Exemplary Needle Variations

The following descriptions relate to various needles that may be incorporated into biopsy device (10) as a substitute for needle (20) described above. While the following is provided as a collection of separate examples, it should be understood that any or all of the features of the below described examples may be varied, modified, substituted, supplemented, or combined in any suitable fashion. In other words, the teachings from one or more of the below examples may be readily combined and/or interchanged with the teachings of one or more other examples described below. The following examples therefore should not be viewed in isolation from each other. The following examples should also not be viewed as exhaustively setting forth the types of features that may be incorporated into the needle of a biopsy device. The various needle features described below may be varied, modified, substituted, supplemented, or combined in various ways as will be apparent to those of ordinary skill in the art in view of the teachings herein.

A. Exemplary Needle with Dimpled Cannula

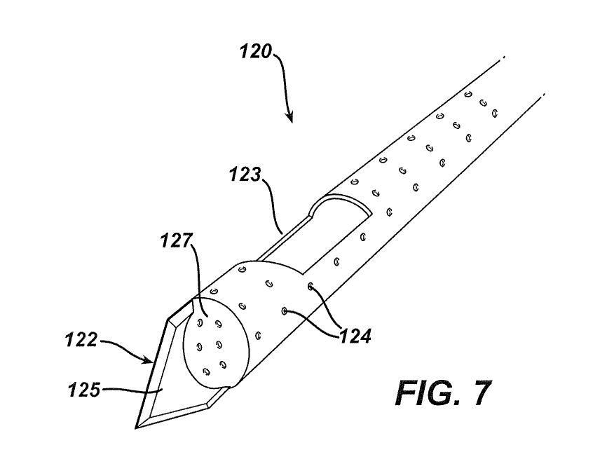

FIG. 7 depicts an exemplary alternative version of a needle (120). Needle (120) of this example has a lateral aperture (123) and a tissue piercing tip (122). Lateral aperture (123) extends longitudinally along needle (120), but in some versions, lateral aperture (123) may comprise any shape or configuration that one of ordinary skill in the art would find suitable in view of the teachings herein. Tissue piercing tip (122) comprises a sharp blade (125) that is configured to pierce and penetrate tissue, as well as a rounded portion (127) adjacent to blade (125). The outer surface of needle (120) comprises a plurality of dimples (124). Dimples (124) are also formed in rounded portion (127). Dimples (124) of the present example are formed as recesses in the exterior of needle (120) and rounded portion (127), but dimples (124) do not form openings through the sidewall of needle (120) or rounded portion (127). For instance, fluids such as liquid or air, etc., cannot pass through the sidewall of needle (120) or rounded portion (127) via dimples (124) in the present example. Dimples (124) may be formed using any suitable process or combination of processes. By way of example only, dimples (124) may be formed through grinding, milling, shot peening, etc.

In the illustrated version, dimples (124) are distributed uniformly across the outer surface of needle (120). However, in some versions, dimples (124) may be distributed in predefined clusters or in predefined patterns as one of ordinary skill in the art would find suitable in view of the teachings herein. For example, dimples (124) may be more heavily concentrated in one portion of the surface of needle (120) than another portion (e.g., more concentrated in the distal portion of needle (120) than in proximal portion of needle (120), etc.).

Each of dimples (124) has a generally concave, circular shape. However, in some versions, each of dimples (124) may have a shape other than a circular shape. It will be appreciated that the shape of dimples (124), which in the illustrated version is concave and circular, may aid in the visibility, such as through higher contrast, of needle (120) when viewed under ultrasound. In other words, dimples (124) may provide greater echogenicity than a smooth surface that might otherwise be found on the exterior of needle (120). It will therefore be appreciated that, for example, dimples (124) of needle (120) may better show an outline of the surface of needle (120) so that needle (120) may be safely guided and precisely positioned within tissue. It will also be appreciated that dimples (124) may reduce drag force of needle (120) against tissue when needle (120) is inserted into the breast of a patient. A needle (120) with dimples (124) may thus require less force to penetrate tissue as compared with an otherwise similar needle (20) that lacks dimples (124).

In some versions, for example, each of dimples (124) may have a square shape, diamond shape, pyramid shape, elliptical shape, crescent shape, or any other shape as one of ordinary skill in the art would find suitable in view of the teachings herein. In some versions, a portion of dimples (124) may have one predetermined shape, such as circular, whereas another portion of dimples (124) may have a different shape, such as triangular. Additionally, each of dimples (124) may be shaped to be convex (i.e., as protrusions) rather than concave (i.e., as recesses). In some versions, a portion of dimples (124) may have a convex shape and another portion of dimples (124) may have a concave shape. Any suitable combination of shapes and selection of convex or concave for dimples (124) may be used.

As shown in FIG. 7, dimples (124) do not cover tissue piercing tip (122) in the present example. In other words, dimples (124) are not formed in blade (125) in this example. However in some other versions, as will be described below, dimples (124) may cover tissue piercing tip (122).

Needle (120) shown in FIG. 7 may be used in a variety of suitable ways as will be apparent to one of ordinary skill in the art in view of the teachings herein. For example, needle (120) may be inserted into the breast of a patient. An ultrasound imaging device may be used to view the interior portion of the breast with needle (120) inside of breast tissue, such as to view the position of needle (120) in relation to a lesion of interest. Dimples (124) may act to deflect ultrasound waves, thereby providing a better image of needle (120) within the breast. As needle (120) is advanced within the breast, the user may be able to better determine the precise positioning of needle (120) as a result of dimples (124) providing better contrast of needle (120) under ultrasound. Once a tissue sample of the breast is removed, needle (120) may be removed from the breast. The user may use the image provided by the ultrasound to monitor the path of needle (120) as it is being removed from the breast.

B. Exemplary Needle with Dimpled Cannula and Serrated Edge

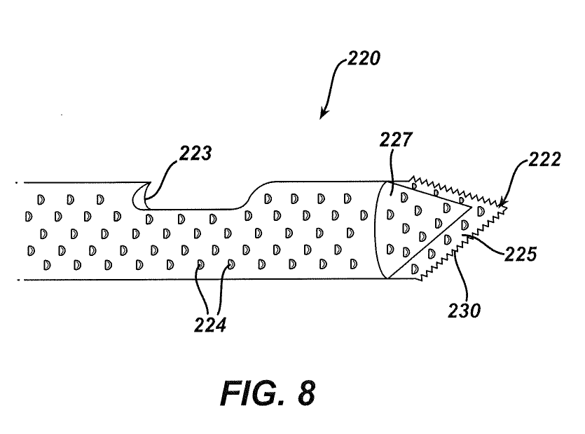

FIG. 8 depicts another version of an exemplary needle (220). Needle (220) of this example has a lateral aperture (223) and a tissue piercing tip (222). Tissue piercing tip (222) comprises a sharp blade (225) that is configured to pierce and penetrate tissue, as well as a rounded portion (227) adjacent to blade (225). Blade (225) comprises a serrated edge (230). Serrated edge (230) runs along the upper and lower lengths of the edge of blade (225). The serrations of serrated edge (230) provide a sawtooth profile in the present example, though it should be understood that the serrations may have any other suitable profile. Serrated edge (230) may be formed in blade (225) in a variety of ways. For instance, serrated edge (230) may be formed through stamping or die-cutting, grinding, milling, laser cutting, and/or using any other suitable process, including combinations of processes. It should also be understood that, in versions where blade (225) is formed separate from needle (220) and is later secured to needle (220) to provide piercing tip (222), serrations in serrated edge (230) may be formed before and/or after blade (225) is secured to needle (220).

In some versions, serrated edge (230) increases contrast of the distal edge of tissue piercing tip (222) in an ultrasound image. Needle (220) also comprises a plurality of dimples (224) positioned on the surface of needle (220) and on the surface of rounded portion (227). Dimples (224) of this example are similar to dimples (124) described above. In the illustrated version, dimples (224) are formed along the entire exterior surface of needle (220). Dimples (224) further extend to tissue piercing tip (222) in this example, including the sides of blade (225) in addition to rounded portion (227). In other words, the sides of blade (225) include dimples (224) in this example, though dimples (224) may be omitted from blade (225) if desired. As further merely illustrative variations, dimples (224) in blade (225) may be substituted or supplemented with protrusions, through holes, and/or various other features.

Needle (220) shown in FIG. 8 may be used in a variety of suitable ways as will be apparent to one of ordinary skill in the art in view of the teachings herein. For example, needle (220) may be inserted into the breast of a patient. An ultrasound imaging device may be used to view the interior portion of the breast with needle (220) inside of breast tissue, such as to view the position of needle (220) in relation to a lesion of interest. Dimples (224) may act to deflect ultrasound waves, thereby providing a better image of needle (220) within the breast. Serrated edge (230) may further act to deflect ultrasound waves, thereby providing a more defined distal edge when viewed under ultrasound. As needle (220) is advanced within the breast, the user may be able to better determine the precise positioning of needle (220) as a result of dimples (224) and serrated edge (230) providing better contrast of needle (220) under ultrasound. Once a tissue sample of the breast is removed, needle (220) may be removed from the breast. The user may use the image provided by the ultrasound to monitor the path of needle (220) as it is being removed from the breast. It will also be appreciated that serrated edge (230) and/or dimples (224) may reduce drag force of needle (220) against tissue when needle (220) is inserted into the breast of a patient. A needle (220) with serrated edge (230) and/or dimples (224) may thus require less force to penetrate tissue as compared with an otherwise similar needle (20) that lacks serrated edge (230) and/or dimples (224).

C. Exemplary Needle with Tip Having Transverse Protrusions

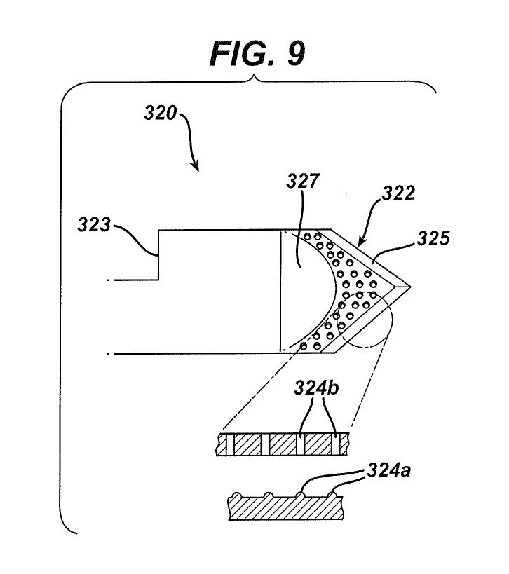

FIG. 9 depicts yet another exemplary version of a needle (320). Needle (320) of this example has a lateral aperture (323) and a tissue piercing tip (322). Tissue piercing tip (322) comprises a sharp blade (325) that is configured to pierce and penetrate tissue, as well as a rounded portion (327) adjacent to blade (325). In the present example, blade (325) comprises a plurality of protrusions (324a) extending outwardly and transversely from blade (325). Protrusions (324a) are limited to tissue piercing tip (322) and do not extend along the surface of the length of needle (320) in this example. Of course, protrusions (324a) may extend along any suitable part of the length of needle (320) as desired. In addition to or in lieu of including protrusions (324a), blade (325) may include a plurality of through holes (324b). Holes (324b) may have a circular shape and may be uniformly spaced throughout tissue piercing tip (322). In other versions, holes (324b) may have any other suitable shape such as, for example, square shaped, triangular, elliptical, crescent shaped, and/or any other shape as would be suitable to one of ordinary skill in the art in view of the teachings herein. In still other versions, protrusions (324a) are substituted or supplemented with coined-in dimples (not shown) and/or other features. In addition or in the alternative, rounded portion (327) may have dimples or other types of indentations (e.g., coined features), protrusions, and/or other features, if desired.

It should be understood that protrusions (324a) and/or holes (324b) may provide better visibility of tip (322) under ultrasound than tip (322) might otherwise have in the absence of protrusions (324a) and/or holes (324b). Protrusions (324a) and/or holes (324b) may thus facilitate guidance of tip (322) through tissue (e.g., breast tissue) to more accurately position aperture (323) adjacent to a lesion under ultrasound visualization. It should also be understood that protrusions (324a) and/or holes (324b) may reduce drag force of needle (320) against tissue when needle (320) is inserted into the breast of a patient. A needle (320) with protrusions (324a) and/or holes (324b) may thus require less force to penetrate tissue as compared with an otherwise similar needle (20) that lacks protrusions (324a) and/or holes (324b).

D. Exemplary Needle with Scalloped Cannula and Multi-Faceted Tip

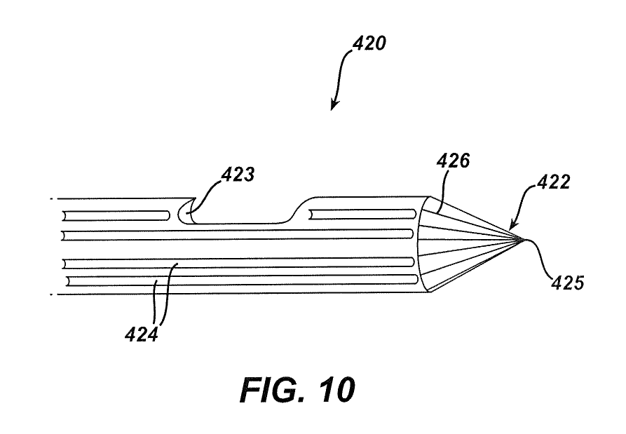

FIG. 10 depicts yet another version of an exemplary needle (420). Needle (420) of this example has a lateral aperture (423) and a tissue piercing tip (422). In the illustrated version, needle (420) comprises a plurality of scallops (424) extending longitudinally along the length of needle (420). Scallops (424) are positioned substantially uniformly around the circumference of needle (420). Scallops (424) may further be of any suitable length. For example, some portion or all scallops (424) may span the entire length of needle (420) while some portion of scallops (424) may span only a portion of the entire length of needle (420). Of course, any suitable configuration for scallops (424) may be used, such as a spiral configuration, a cross hatching configuration, or any other suitable configuration as will be apparent to one of ordinary skill in the art in view of the teachings herein. In some other alternative versions, angular grooves may be used in conjunction with or in place of scallops (424).

Tissue piercing tip (422) of the present example comprises a multi-faceted tip having a plurality of facets (426). Facets (426) are circumferentially positioned about tissue piercing tip (422), extending from sharp point (425) of needle (420) to the cannula of needle (420). Facets (426) may generally comprise a series of plates facing a variety of directions, in which the series of plates may form a symmetrical or asymmetrical pattern. In some versions, facets (426) simply comprise flat surfaces presented by a solid material (e.g., metal, ceramic, etc.) forming tip (422).

In versions where facets (426) are in an asymmetrical pattern, such an asymmetrical pattern may be used to provide for a rotational key such that the rotational key may be used to determine the rotational orientation of tissue piercing tip (422) when viewed under ultrasound. Such rotational orientation of tissue piercing tip (422) may indicate the rotational orientation of aperture (423). Thus, facets (426) may be viewed under ultrasound while needle (420) is in tissue to assist in positioning aperture (423) adjacent to a lesion of interest. In addition to providing a reference of the rotational position of aperture (423), it will be appreciated that facets (426) and scallops (424) may improve the quality of images of needle (420) when used in conjunction with an ultrasound imaging device. It will also be appreciated that facets (426) and/or scallops (424) may reduce drag force of needle (420) against tissue when needle (420) is inserted into the breast of a patient. A needle (420) with facets (426) and/or scallops (424) may thus require less force to penetrate tissue as compared with an otherwise similar needle (20) that lacks facets (426) and/or scallops (424).

E. Exemplary Needle with Dimpled Tip and Serrated Edge

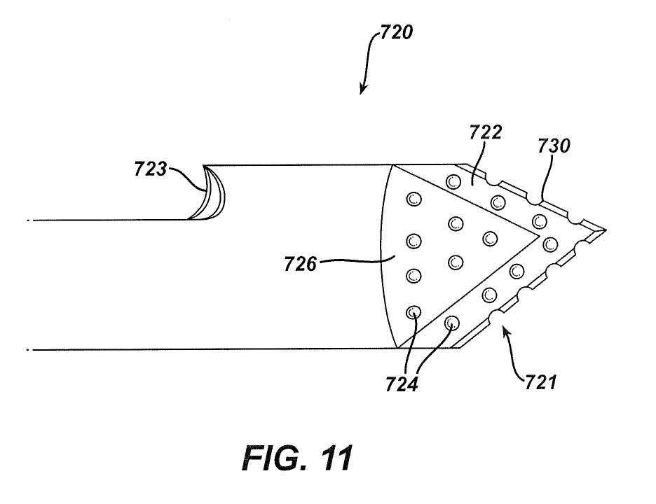

FIG. 11 depicts another version of an exemplary needle (720). Needle (720) of this example has a lateral aperture (723) and a tissue piercing tip (721). Tissue piercing tip (721) comprises a tip body (726) and a blade (722). Tip body (726) has a generally conical shape so as to facilitate insertion into tissue in conjunction with blade (722). Of course, tip body (726) may have any other suitable configuration. Blade (722) is held by tip body (726) so as to point outward in the direction needle (720) is to be inserted into tissue. Blade (722) generally follows the upper and lower contour of tip body (726), but any suitable shape for blade (722) may be used as would be apparent to one of ordinary skill in the art in view of the teachings herein. Blade (722) comprises a serrated edge (730). Serrated edge (730) comprises a series of alternating grooved portions and straight blade portions. Both the grooved and straight blade portions may be sharpened to slice through tissue, or alternatively, either or both portions may be non-sharpened. As can be seen in FIG. 11, the grooved portions of serrated edge (730) are round in the present example. By way of example only, the grooved portions may be configured like partial circles having a constant and consistent radius of curvature. Such round grooves may provide surprisingly ideal echogenicity in certain examples. In some other versions, the grooved portions are triangular or jagged, providing serrated edge (730) with a configuration that is saw-like. Of course, any other suitable configuration may be used. In some versions, the upper portion of serrated edge (730) has four grooves while the lower portion of serrated edge (730) also has four grooves. Alternatively, any suitable number of grooves may be provided. It should also be understood that the upper portion of serrated edge (730) have a different number of grooves than the lower portion of serrated edge (730).

It should be understood that, as tip (721) is inserted into tissue and after tip (721) is inserted in tissue, an ultrasound device may be used in conjunction with needle (720). In particular, the ultrasound device may be used to monitor the advancement of needle (720) through tissue and detect the position of needle (720) relative to a suspect lesion within the tissue. In the present example, serrated edge (730) facilitates deflection of ultrasound signals as needle (720) advances through tissue, thereby improving contrast and visibility of serrated edge (730). In other words, the presence of and configuration of serrated edge (730) may provide greater visibility of tip (721) within tissue under ultrasound imaging than would otherwise be obtained in the absence of serrated edge (730).

Furthermore, tissue piercing tip (721) of the present example includes a plurality of dimples (724). Dimples (724) cover tip body (726) and portions of blade (722) that are proximal to serrated edge (730). Dimples (724) may cover serrated edge (730) and tip body (726) in a substantially uniform manner, or alternatively, dimples (724) may be spaced apart in a non-uniform manner. It should also be understood that dimples (724) may be provided only on tip body (726), be provided only on blade (722), or be omitted altogether, if desired. Dimples (724) may be configured like any other dimples described herein. Similarly, dimples (724) may provide enhanced visibility of needle (720) within tissue under ultrasound imaging like any other dimples or similar features described herein.

As yet another merely illustrative variation, needle (720) may be configured such that tip body (726) includes dimples (724), such that blade (722) includes serrated edge (730) with grooved portions, and such that blade (722) includes through holes formed through portions of blade (722) that are proximal to serrated edge (730). While such through holes are not shown in FIG. 11, it should be understood that such through holes may be formed similar to through holes (324b) described above and shown in FIG. 9. Alternatively, such through holes may have any other suitable configuration. Still other suitable features and configurations that needle (720) may have will be apparent to those of ordinary skill in the art in view of the teachings herein.

In some versions, dimples (724) and the grooved portions of serrated edge (730) are formed using the same instrument. In particular, in some such versions, a drill bit or similar type of milling tool is used to form dimples (724) (e.g., without actually forming holes through needle (720), etc.), and that same drill bit or other type of milling tool is used to form the grooved portions of serrated edge (730). To the extent that blade (722) has through holes, that same drill bit or other type of milling tool may also be used to form such through holes. Of course, needle (720) may be formed using any other suitable techniques or combinations of techniques, including but not limited to stamping or die-cutting, grinding, milling, laser cutting, etc.

F. Exemplary Needle with Grooved Cutter

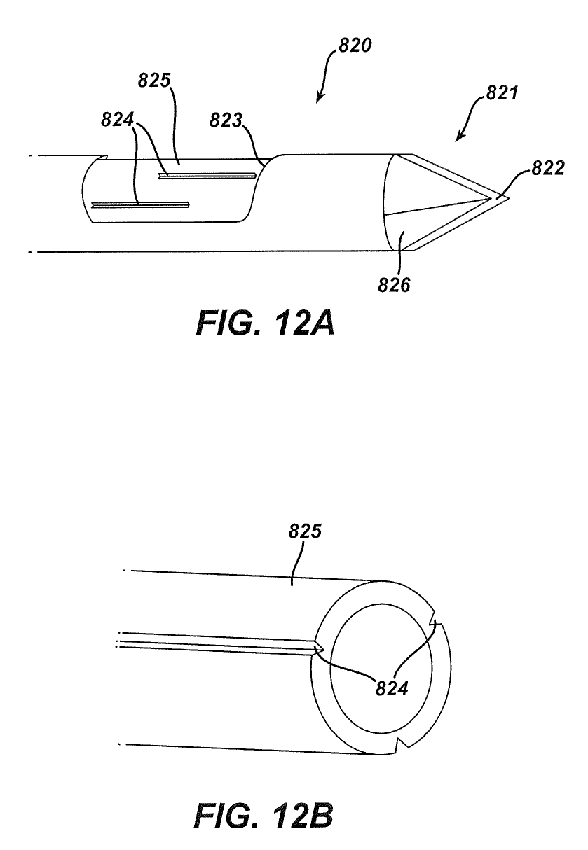

FIG. 12A depicts yet another version of an exemplary needle (820). Needle (820) of this example has a lateral aperture (823) and a tissue piercing tip (821). Tissue piercing tip (821) comprises a tip body (826) and a blade (822). Lateral aperture (823) extends longitudinally along part of the length of needle (820). The surface of needle (820) is generally smooth in the present example, though it should be understood that needle (820) may include a textured or dimpled surface and/or a variety of other features. A cutter (825) extends through a lumen defined by needle (820). Cutter (825) comprises a distal cutting end that cuts tissue drawn into lateral aperture (823) as cutter (825) advances distally and/or rotationally through needle (820). FIG. 12B depicts a cross sectional view of cutter (825) of the present example. Cutter (825) comprises a plurality of longitudinally extending grooves (824). While three grooves (824) are shown, it should be understood that any suitable number of grooves (824) may be used, including but not limited to a single groove (824), two grooves (824), more than three grooves (824), etc.

Grooves (824) have generally a V-shape in the present example, though it should be understood that any other suitable shape or combination of shapes may be used. For example, rectangular or circular shaped grooves (824) may be used. In the illustrated version, grooves (824) are oriented parallel to one another. In addition, grooves (824) are generally spaced apart around the circumference of cutter (825) such that grooves (824) are uniformly spaced apart. However, grooves (824) may be spaced apart such that one area of cutter (825) comprises more grooves (824) than another area. Grooves (824) are further positioned such that grooves (824) are parallel to the longitudinal axis of cutter (825), but any other suitable positioning and orientation of grooves (824) on cutter (825) may be used. Grooves (824) do not extend along the entire length of cutter (825) in the example depicted in FIG. 12A, but in some versions, grooves (824) may extend along the entire length of cutter (825). In the illustrated version, grooves (824) are staggered along the length of cutter (825) and have lengths that are less than the length of cutter (825); and in some versions, grooves (824) may have a length shorter than the length of lateral aperture (823).

As cutter (825) rotates and/or advances, the user may use ultrasound imaging to determine the position of needle (820) within tissue. Grooves (824) may deflect ultrasound signals such that the deflection of ultrasound signals by grooves (824) may provide better contrast and/or visibility of cutter (825) within needle (820). It will be appreciated that due to grooves (824) having a different textured surface in relation to needle (820), cutter (825) may be distinguishable from needle (820) when viewed under ultrasound such that under ultrasound, cutter (825) can be seen advancing within needle (820). It should also be understood that, when cutter (825) is advanced distally relative to needle (820), grooves (824) that are exposed through lateral aperture (823) may facilitate location of lateral aperture (823) under ultrasound imaging, due to the increased echogenicity provided by grooves (824), thereby facilitating positioning of lateral aperture (823) adjacent to a lesion of interest. Furthermore, grooves (824) positioned asymmetrically about the circumference of cutter (825) may be used to determine the rotational orientation of cutter (825). The length of grooves (824) may also be used to provide information to the user regarding the degree of advancement of cutter (825) within needle (820). Any other useful information that the position and orientation of grooves (824) may be provided to the user may be considered when positioning grooves (824) on cutter (825) as would be apparent to one of ordinary skill in the art in view of the teachings herein.

G. Exemplary Needle with Coated Cannula

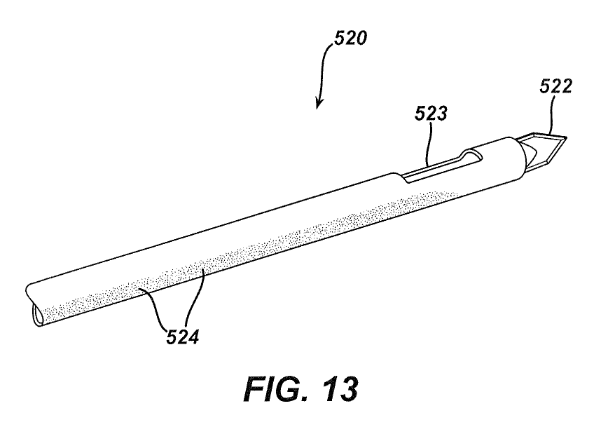

FIG. 13 depicts yet another version of an exemplary needle (520). Needle (520) of this example has a lateral aperture (523) and a tissue piercing tip (522). Needle (520) further comprises an outer surface that is coated with a coagulant (524). Coagulant (524) may be applied to the outer surface of needle (520) uniformly, or alternatively, coagulant (524) may be applied such that only a portion of the outer surface of needle (520) is covered with coagulant (524). For example, half of needle (520) may be coated with coagulant (524) or coagulant (524) may be applied, for example, to a circular portion of the outer surface of needle (520).

In some versions, coagulant (524) may be applied more heavily to some portions of needle (520) relative to other portions of needle (520). In some versions, coagulant (524) may be applied to needle (520) prior to insertion of needle (520) into the breast. In some other versions, coagulant (524) may be applied to needle (520) after needle (520) is inserted into the breast by, for example, injecting coagulant (524) onto the surface of needle (520). It will be appreciated that use of coagulant (524) may be used to cause tissue to coagulate as needle (520) is inserted or removed through the tissue. In addition or in the alternative, a coagulant (524) may prevent or reduce the infiltration of blood and/or other bodily fluids from entering lateral aperture (523).

Additionally, the inner surface of cutter (50) may be coated with a lubricating surfactant, such as, for example, tribofilm. It will be appreciated that application of a lubricating surfactant may allow better transportation of samples proximally through the lumen (52) of cutter (50) to a collection chamber (not shown). The lubricating surfactant may be applied along the entire length of the inner surface of cutter (50) or may alternatively be applied to portions of inner surface of cutter (50) as one of ordinary skill in the art would find suitable in view of the teachings herein.

H. Exemplary Needle with Rotating Tip

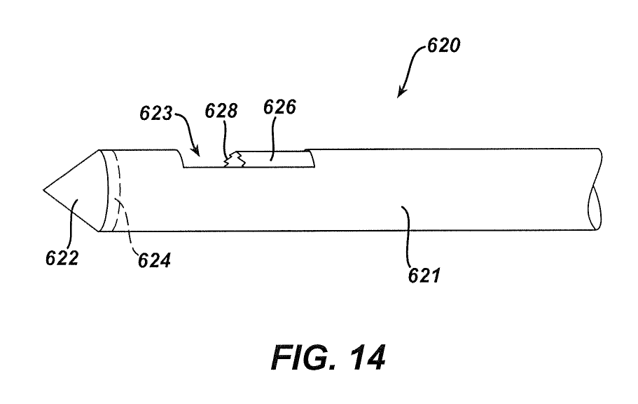

FIG. 14 depicts a yet another version of an exemplary needle (620). Needle (620) of this example has a lateral aperture (623), a tissue piercing tip (622), and a cannula (621). Tissue piercing tip (622) is secured to cannula (621) by a coupling (624), which allows tip (622) to rotate relative to cannula (621). A cutter (626) extends through a lumen defined by cannula (621). Cutter (626) of this example includes a serrated distal edge (628). A proximal face (not shown) of piercing tip (622), which is disposed within cannula (621), includes recesses that complement the serrations of distal edge (628). In particular, when cutter (626) is advanced to a distal position, distal edge (628) engages the proximal face of piercing tip (622), with serrations of distal edge (628) engaging the complementary recesses in the proximal face of piercing tip (622). With tip (622) and cutter (626) so engaged, rotation of cutter (626) causes corresponding rotation of tip (622). Cutter (626) and tip (622) may thus rotate concomitantly relative to cannula (621). While distal edge (628) has serrations in the present example, various other suitable configurations for distal edge (628) will be apparent to those of ordinary skill in the art in view of the teachings herein.

Tissue piercing tip (622) may comprise a smooth surface having a low coefficient of friction so as to allow easier insertion into the breast. Alternatively, tissue piercing tip (622) may comprise a textured or patterned surface such that tissue piercing tip (622) may more easily tunnel through breast tissue to advance within the breast. For example, tissue piercing tip (622) may comprise a spiral patterned surface or a multi-faceted surface. In some versions, tissue piercing tip (622) may comprise a dimpled surface with a plurality of dimples around the circumference of tissue piercing tip (622). It will be appreciated that in some configurations, the surface of tissue piercing tip (622) may adhere or exhibit some adherence to tissue as tissue piercing tip (622) is advanced within tissue. The plurality of dimples, in contrast, may reduce or eliminate the adherence caused by the surface of tissue piercing tip (622), thereby aiding in advancing of needle (620) into tissue such that less force may be used. Optional features of tip (622) (e.g., any of the various biopsy needle tip features described herein) may also provide better visibility of tip (622) under ultrasound imaging.

Needle (620) shown in FIG. 12 may be used in a variety of suitable ways that may be apparent to one of ordinary skill in the art in view of the teachings herein. For example, an incision may be made in the breast so as to provide needle (620) with an insertion point. Alternatively, tissue piercing tip (622) may be sharp enough so as to pierce the breast without the use of an advance incision. In either case, cutter (626) may be engaged with tip (622) and rotating as needle (620) is advanced in tissue. The resulting rotation of tip (622) may help needle (620) bore through tissue, thereby reducing the insertion force required from the user in order to penetrate the tissue. As needle (620) is advancing into the breast, an ultrasound imaging device (not shown) may be used to determine the positioning of needle (620) within the tissue. Once needle (620) and tissue piercing tip (622) have reached an appropriate position as determined by the user, cutter (626) may be retracted proximally to allow a portion of tissue to enter lateral aperture (623). Once tissue enters lateral aperture (623), cutter (626) be advanced distally within cannula (621) (and may also rotate during such advancement, if desired) to sever tissue protruding through lateral aperture (623). If desired, the user may actuate the cutter several times to acquire multiple samples of tissue. In some such versions, the degree of distal advancement of cutter (626) within needle (620) may be restricted after needle (620) has been inserted in tissue. For instance, cutter (626) may be controlled such that it stops just short of engaging the proximal face of tip (622) once needle (620) has reached a sufficient depth within tissue.

In some versions, a vacuum port may be in communication with cannula (621) thereby communicating a vacuum to lateral aperture (623), which may assist in drawing tissue into lateral aperture (623) for cutting. Once a sufficient number of tissue samples have been taken, needle (620) may be withdrawn from the tissue and removed through the incision or opening that needle (620) entered the tissue. As needle (620) is removed from the tissue, tissue piercing tip (622) may again be rotated, if desired. Alternatively, tip (622) need not be rotated as needle (620) is withdrawn from tissue.

While rotation of tip (622) is provided through rotation of cutter (626) in the present example, it should be understood that tip (622) may be rotated in a variety of other ways. By way of example only, a separate motor may be located at or near the distal end of cannula (621), with such a motor being operable to rotate tip (622). As yet another merely illustrative example, a rotary drive shaft may extend through cannula (621) and may be coupled with tip (622) to rotate tip. Such a rotary drive shaft may also extend through the lumen of cutter (626). Still other suitable ways in which tip (622) may be rotated will be apparent to those of ordinary skill in the art in view of the teachings herein.

I. Exemplary Needle with Blade Having Oblong Serrations

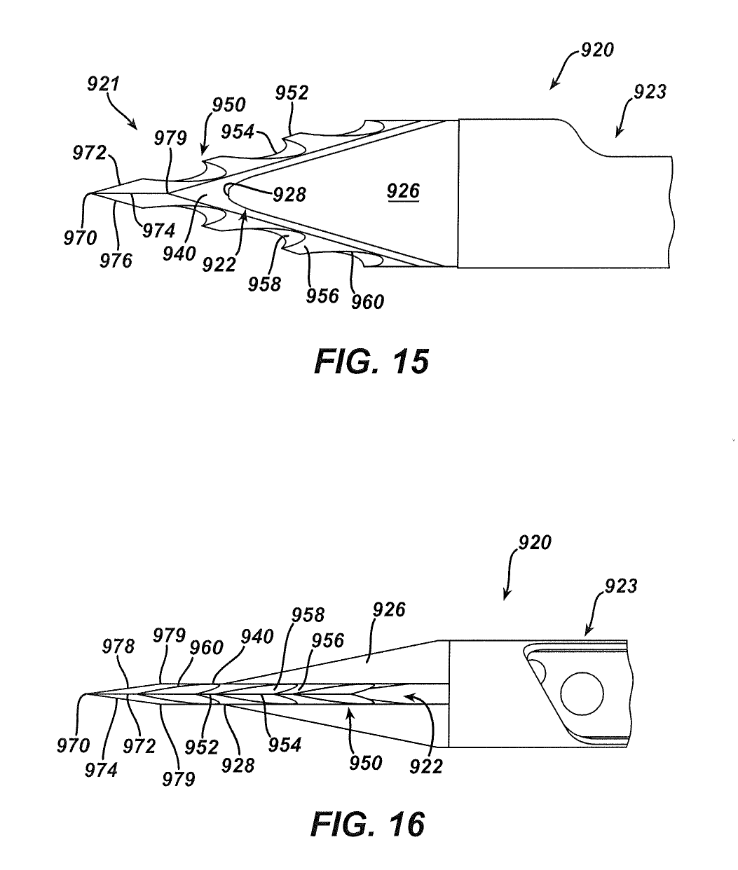

FIGS. 15-16 depict another version of an exemplary needle (920). Needle (920) of this example has a lateral aperture (923) and a tissue piercing tip (921). Tissue piercing tip (921) comprises a tip body (926) and a blade (922). Tip body (926) has a generally tapered shape so as to facilitate insertion into tissue in conjunction with blade (922). Of course, tip body (926) may have any other suitable configuration. Blade (922) is held by tip body (926) so as to point outward in the direction needle (920) is to be inserted into tissue. Blade (922) of this example comprises a substantially flat exposed portion (940), a plurality of serrations (950), and a sharp distal point (970).

Serrations (950) of the present example comprise a plurality of straight sharp edges (952) and a plurality of curved sharp edges (954). As best seen in FIG. 16, a pair of substantially flat yet angled faces (956) converge at each straight sharp edge (952). A pair of concave faces (958) converge at each curved sharp edge (954). In addition, each concave face (958) laterally terminates at a respective outer sharp edge (960). As best seen in FIG. 15, distal portions of each outer sharp edge (960) extend along a path that is substantially parallel to the longitudinal axis defined by needle (920). Of course, any other suitable configuration may be used. In some alternative versions, serrations (950) are provided in just one lateral side of blade (922) in a chisel ground fashion, such that the other lateral side of blade (922) is simply flat. For instance, a chisel ground version of blade (922) may lack angled faces (956) and concave faces (958) along an entire side of blade (922), which would simply be flat on that side. Other suitable configurations will be apparent to those of ordinary skill in the art in view of the teachings herein. It should also be understood that any blade disclosed herein may have a chisel ground configuration, if desired.

In the present example, as best seen in FIG. 15, serrations (950) are provided along both the top side and along the bottom side of blade (922). In some other versions, serrations (950) are provided only along the top side of blade (922), with the bottom side of blade (922) having a single straight cutting edge. As yet another merely illustrative variation, serrations (950) may be provided only along the bottom side of blade (922), with the top side of blade (922) having a single straight cutting edge.

In the present example, concave faces (958) present an oblong curvature, similar to an ellipse, though it should be understood that any suitable curvature may be used. As can be seen in FIGS. 15-16, the pitch defined by pairs of concave faces (958) at each corresponding curved sharp edge (954) is steeper than the pitch defined by pairs of angled faces (956) at each corresponding straight sharp edge (952), providing serrations (950) with an oblong scalloped configuration when viewed from the top and from the side. In some versions, concave faces (958) and/or curved sharp edges (954) are formed using a bullet grinder or similar device providing a “hollow ground” configuration. Of course, any other suitable devices or methods may be used to form concave faces (958) and/or curved sharp edges (954), including but not limited to a wire EDM process. In the present example, blade (922) comprises three scallops in its top portion and three scallops in its bottom portion. It should be understood, however, that any other suitable number of scallops may be provided in the top and bottom portions of blade (922), including but not limited to one scallop, two scallops, four scallops, or five scallops.

Sharp distal point (970) is formed by the convergence of sharp edges (972, 974, 976, 978). Sharp edges (972, 974, 976, 978) comprise an upper sharp edge (972), a first side sharp edge (974), a lower sharp edge (976), and a second side sharp edge (978). Edges (972, 974, 976, 978) thus provide the distal end of blade (922) with a multi-faceted configuration (e.g., four converging faces at different orientations) in the present example.

As best seen in FIG. 16, straight sharp edges (952) on the top portion of blade (922), curved sharp edges (954) on the top portion of blade (922), and upper sharp edge (972) are all substantially aligned, such that they all collectively form a single cutting edge. Similarly, straight sharp edges (952) on the bottom portion of blade (922), curved sharp edges (954) on the bottom portion of blade (922), and lower sharp edge (976) are all substantially aligned, such that they also all collectively form a single cutting edge. Side sharp edges (974, 978) simply angle inwardly from flat exposed portion (940) of blade (922). Each side sharp edge (974, 978) proximally terminates at a respective proximal termination point (979). Flat exposed portion (940) distally terminates at the same point (979). Tip body (926) distally terminates at a distal termination point (928) in the present example, the distance along the longitudinal dimension from distal point (970) to proximal termination point (979) is greater than the distance along the longitudinal dimension from proximal termination point (979) to distal termination point (928). Of course, these distances may be the same or may have any other suitable relationship with each other in other versions.

In some versions, the configuration of serrations (950) and/or other features of blade (922) provide greater echogenicity of tip (921) than would be found in a conventional biopsy device needle tip. For instance, the angle and/or shape of concave faces (958) may reflect ultrasound imaging waves better than a pair of substantially flat opposing blade surfaces would. For instance, the scallops of blade (922) may appear brightly and prominently under ultrasound imaging when needle (920) is inserted in tissue at various orientations, allowing a user to detect the position of needle (920) in tissue with relative ease. The multi-faceted configuration of the distal end of blade (922) provided by edges (972, 974, 976, 978) may also reflect ultrasound imaging waves better than a simple pair of opposing flat blade faces would. In addition or in the alternative, the configuration of serrations (950) and/or other features of blade (922) may require less force to be used in order for tip (921) to penetrate tissue than would be required by a conventional biopsy device needle tip. Of course, the various features of blade (922) described herein may provide other results in addition to or in lieu of providing greater echogenicity and/or reduced force to penetrate. It should also be understood that blade (922) may be modified or varied in numerous ways, as will be apparent to those of ordinary skill in the art in view of the teachings herein,

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

Embodiments of the devices disclosed herein can be designed to be disposed of after a single use, or they can be designed to be used multiple times. Embodiments may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A biopsy device comprising:
(a) an elongate needle, wherein the needle comprises a proximal end and a distal end, wherein the needle further comprises a lateral aperture, wherein at least a portion of the distal end of the needle comprises a dimpled surface, wherein the dimpled surface comprises dimples that are sized, spaced, and configured to be echogenic, wherein the distal end of the needle further comprises a piercing tip;
(b) a cylindrical cutter disposed within the needle, wherein the cutter is configured to translate to cut across the lateral aperture; and
(c) a handpiece, wherein the needle extends distally from the handpiece, wherein the handpiece is operable to actuate the cutter.

2. The biopsy device of claim 1, wherein the dimples comprise a plurality of concave indentations.

3. The biopsy device of claim 1, wherein the dimples comprise a plurality of convex protrusions.

4. The biopsy device of claim 1, wherein the piercing tip comprises a blade, wherein the blade comprises at least one serrated edge.

5. The biopsy device of claim 4, wherein the blade comprises a plurality of blade dimples.

6. The biopsy device of claim 4, wherein the blade comprises a first plurality of holes formed transversely through the blade.

7. The biopsy device of claim 1, wherein the needle further comprises a cannula portion, wherein the piercing tip is rotatably coupled with the cannula portion, wherein the cutter extends through the cutting portion.

8. The biopsy device of claim 7, wherein the cutter has a distal edge configured to engage a proximal face of the piercing tip, such that the cutter is operable to rotate the piercing tip through rotation of the cutter.

9. The biopsy device of claim 7, wherein the distal edge of the cutter is serrated.

10. The biopsy device of claim 1, wherein the dimpled surface further comprises at least one scallop.

11. A biopsy device, comprising:
(a) a hub;
(b) a needle extending from the hub, the needle comprising a distal portion and a proximal portion, wherein the distal portion of the cannula comprises a piercing tip and a lateral aperture located proximal to the piercing tip, wherein the needle includes one or more echogenic features that are sized, configured, and arranged to stand out in an ultrasound image relative to other portions of the needle; and
(c) a cutter, wherein at least a portion of the cutter extends through the cannula, wherein the cutter is movable relative to the needle to sever tissue protruding through the lateral aperture.

12. The biopsy device of claim 11, wherein the one or more echogenic features comprise a plurality of dimples operable to deflect signals in ultrasound.

13. The biopsy device of claim 11, wherein the piercing tip comprises at least two edges, wherein the at least two edges are positioned to be coplanar, wherein the at least two edges are configured to intersect at a single point, wherein each of the at least two edges are configured to have at least one serrated portion.

14. The biopsy device of claim 13, wherein the serrations comprise a series of alternating grooved portions and straight blade portions.

15. The biopsy device of claim 14, wherein the grooved portions comprise round grooves.

16. The biopsy device of claim 15, wherein the round grooves are configured as partial circles.

17. The apparatus of claim 11, wherein the piercing tip comprises a multifaceted tip.

18. The apparatus of claim 11, wherein the needle is coated with a coagulating agent.

19. A method of performing a biopsy on a breast using a needle and an ultrasound imaging device, wherein the needle comprises a piercing portion, a lateral aperture, one or more echogenic features, and a cutter, the method comprising:
(a) piercing the breast with the piercing portion;
(b) advancing the needle within the breast;
(c) monitoring the position of the needle within the breast with the ultrasound imaging device;
(d) deflecting ultrasound waves emitted by the ultrasound device with the one or more echogenic features;
(e) guiding the piercing portion of the needle with the ultrasound device, wherein the act of guiding is performed at least in part based on visualization of the one or more echogenic features;
(f) receiving at least a portion of breast tissue through the lateral aperture;
(g) actuating the cutter to sever a portion of tissue; and
(h) removing the needle from the breast.

20. The method of claim 19, wherein the one or more echogenic features are located on the piercing portion, wherein the act of guiding comprises visualizing the location of the piercing portion by visualizing the location of the one or more echogenic features on the piercing portion.
